(19) United States
(12) Patent Application Publication
(10) Pub. No.: US 2025/0387071 A1
(43) Pub. Date: Dec. 25, 2025

(54) UTILZING ONE OR MORE SENSORS TO DETECT MUSCLE ACTIVATION

(71) Applicant: Peke Labs Corp., Wilmington, DE (US)

(72) Inventors: Thane Robert Hunt, Plymouth, MA (US); Samuel William Austin Lazarus, Newport, RI (US)

(21) Appl. No.: 19/243,566

(22) Filed: Jun. 19, 2025

Related U.S. Application Data
(60) Provisional application No. 63/662,303, filed on Jun. 20, 2024.

(51) Int. Cl. A61B 5/395

(52) U.S. Cl. CPC A61B 5/395

(57) ABSTRACT

A muscle activation sensing system includes a mechanical sensor mounted on a body part and is configured to detect a change in a muscle associated with the body part. A location of the muscle activation sensing system on the body part is determined. A control signal is derived based on the detected change in the muscle associated with the body part and the determined location. The control signal is outputted to a device.

### CROSS REFERENCE TO OTHER APPLICATIONS

This application claims priority to U.S. Provisional Patent Application No. 63/662,303 entitled UTILIZING ONE OR MORE SENSORS TO DETECT MUSCLE ACTIVATION filed Jun. 20, 2024 which is incorporated herein by reference for all purposes.

### BACKGROUND OF THE INVENTION

An electromyography (EMG) sensor is a device that detects, measures, and records the electrical signals generated by skeletal muscles. These sensors are used in a variety of applications, including assessing muscle and nerve health, controlling robotic limbs through muscle signals, tracking muscle activation and fatigue, and enabling gesture recognition and control systems. However, because EMG signals are very small, they can easily be affected by electrical interference from nearby electronics, power lines, or other sensors. Additionally, movement of the sensor or skin can introduce unwanted noise, leading to inaccurate readings. Lastly, EMG sensors may pick up signals from nearby muscles (not the target muscle), making it difficult to isolate activity from a specific target muscle.

### BRIEF DESCRIPTION OF THE DRAWINGS

Various embodiments of the invention are disclosed in the following detailed description and the accompanying drawings.

FIG. 1A is a perspective view of a muscle activation sensing system in accordance with some embodiments.

FIG. 1B is a top-down view of a muscle activation sensing system in accordance with some embodiments.

FIG. 1C is a perspective view of a portion of a muscle activation sensing system in accordance with some embodiments.

FIG. 2 is a block diagram of a control system in accordance with some embodiments.

FIG. 3 is a block diagram of a muscle-based controller in accordance with some embodiments.

FIG. 4A is a block diagram illustrating an example of a muscle activation sensing system in accordance with some embodiments.

FIGS. 4B and 4C are examples of force readings outputted by a muscle activation sensing system in accordance with some embodiments.

FIGS. 5A-5C are diagrams illustrating an arrangement and mounting of sensors in accordance with some embodiments.

FIGS. 6A & 6B are diagrams illustrating crosstalk being reduced in the muscle activation sensing system in accordance with some embodiments.

FIG. 7 is a diagram illustrating sensing limb size and shape in accordance with some embodiments.

FIG. 8 is a diagram illustrating how the position of the Muscle Activation Sensing System on the arm can be determined in accordance with some embodiments.

FIGS. 9 and 10 are diagrams illustrating how the band angle of the muscle activation sensing system can be determined in accordance with some embodiments.

FIG. 11 is a diagram illustrating how a position of the muscle activation sensing system on the arm can be determined in accordance with some embodiments.

FIG. 12 is a diagram illustrating an imager in accordance with some embodiments.

FIG. 13 is a diagram illustrating an imager and distance sensor in accordance with some embodiments.

FIGS. 14A-C are diagrams illustrating how using anatomical information can be used to aid in algorithm interpretation of user intent in accordance with some embodiments.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

System and methods to detect muscle activation are disclosed herein. A muscle activation sensing system is utilized to measure the mechanical response of a localized, known-size region of muscle tissue. The muscle activation sensing system includes a sensor having an indenter geometry, such as a half-hemispherical protrusion, positioned to interface with the skin surface. When the underlying muscle tissue contracts and stiffens, the muscle applies force against the indenter, displacing it from a recessed position and producing a measurable force signal. This protruding indenter arrangement increases the sensitivity of the sensor to muscle activity. Additionally, muscle contraction may cause the muscle to expand laterally, thereby increasing strap tension and further compressing the sensor.

In some instances, the expansion of the muscle may generate cross-talk by influencing signals from adjacent, non-target muscle regions. To address this, the sensor may be positioned between two adjacent sensors, thereby providing passive shear-force isolation that reduces interference from neighboring muscle activity. Additionally, the system may incorporate techniques for differentially separating the force contributions of the muscle from those resulting from strap tension. Such separation may be accomplished through passive mechanical isolation, for example, by configuring structural components with complementary stiffness properties or incorporating shear-force isolation features. Alternatively, or in combination, active isolation techniques may be employed, such as measuring variations in strap tension or strap length and calculating the resulting forces acting on the sensor. Implementing these isolation techniques offers two advantages: (1) providing reliable measurement of absolute force magnitudes rather than relative or percentage-based variations, and (2) enabling the system to differentiate between forces generated internally by muscle contraction and those caused by external disturbances, such as pulling, squeezing, or other artifacts.

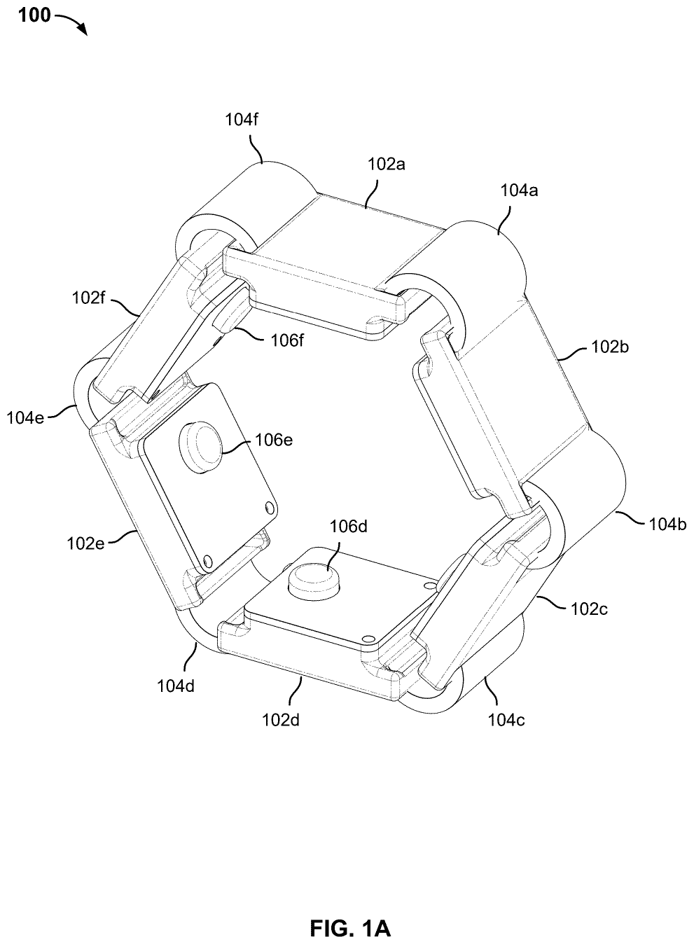

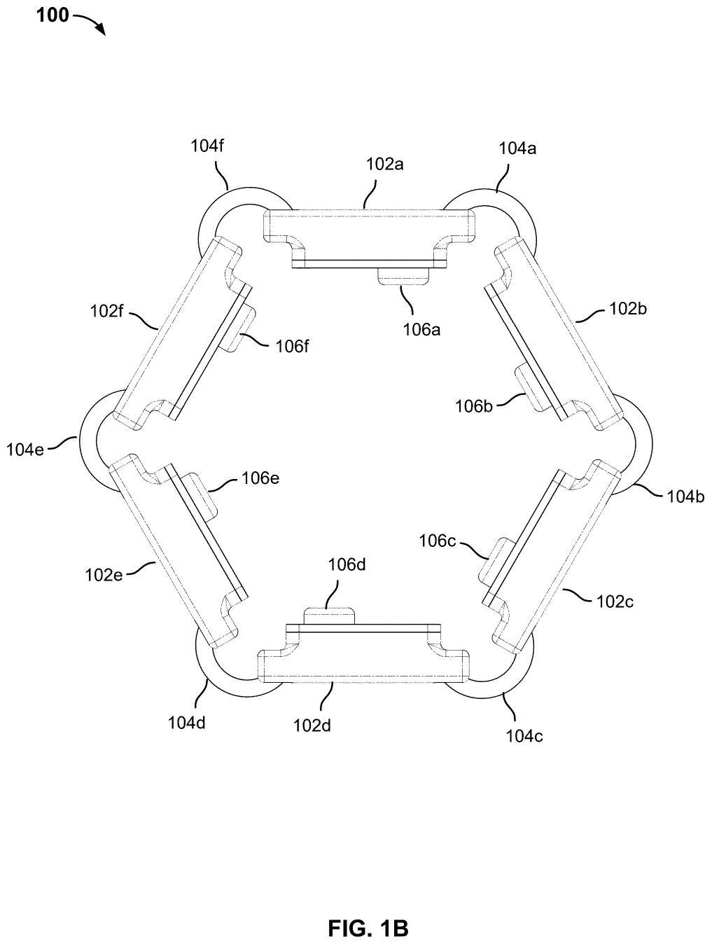

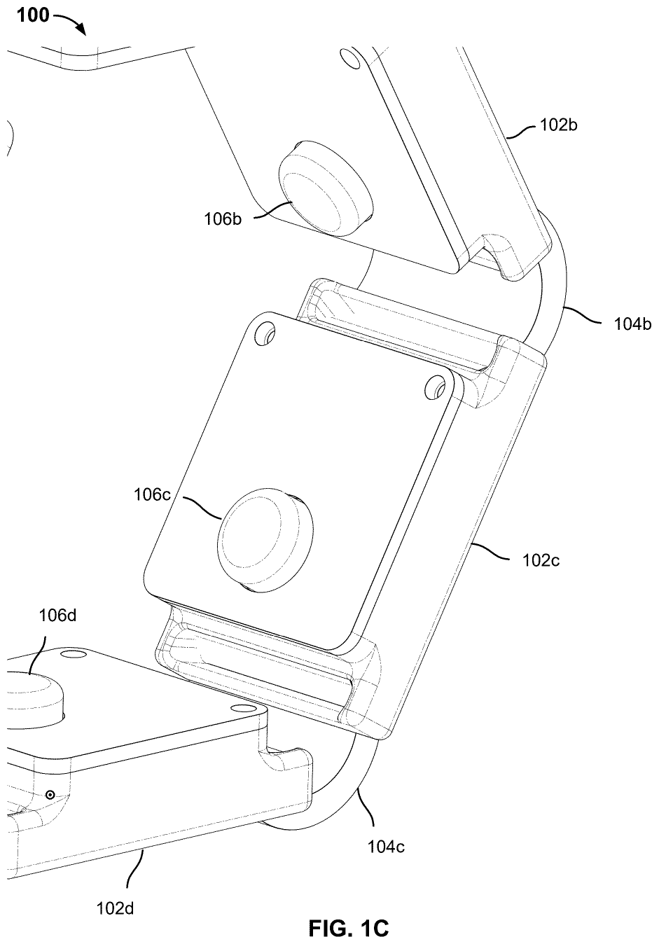

FIG. 1A is a perspective view of a muscle activation sensing system in accordance with some embodiments. FIG. 1B is a top-down view of a muscle activation sensing system in accordance with some embodiments. FIG. 1C is a perspective view of a portion of a muscle activation sensing system in accordance with some embodiments. In the example shown, muscle activation sensing system 100 includes a plurality of housings 102a, 102b, 102c, 102d, 102c, 102f. In some embodiments, the plurality of housings 102a, 102b, 102c, 102d, 102e, 102f are used to house a corresponding force sensor. In some embodiments, the plurality of housings 102a, 102b, 102c, 102d, 102e, 102f are used to house a corresponding displacement sensor. In some embodiments, the plurality of housings 102a, 102b, 102c, 102d, 102e, 102f are used to house a corresponding force sensor, a corresponding displacement sensor, other type of mechanical sensor. Although muscle activation sensing system 100 is shown having six housing for six sensors, muscle activation sensing system 100 may have 1:n housings for 1:n sensors. In some embodiments, a housing includes one or more sensors. In some embodiments, a housing includes multiple sensors of the same type. In some embodiments, a housing includes different types of sensors.

Each of the housings 102a, 102b, 102c, 102d, 102e, 102f include a corresponding body part interface 106a, 106b, 106c, 106d, 106e, 106f. A body part interface contacts a skin portion of a body part.

The plurality of housings 102a, 102b, 102c, 102d, 102e, 102f are connected using links 104a, 104b, 104c, 104d, 104e, 104f. That is, housing 102a and housing 102b are connected to each other via link 104a, housing 102b and housing 102c are connected to each other via link 104b, housing 102c and housing 102d are connected to each other via link 104c, housing 102d and housing 102e are connected to each other via link 104d, housing 102e and housing 102f are connected to each other via link 104e, and housing 102f and housing 102a are connected to each other via link 104f.

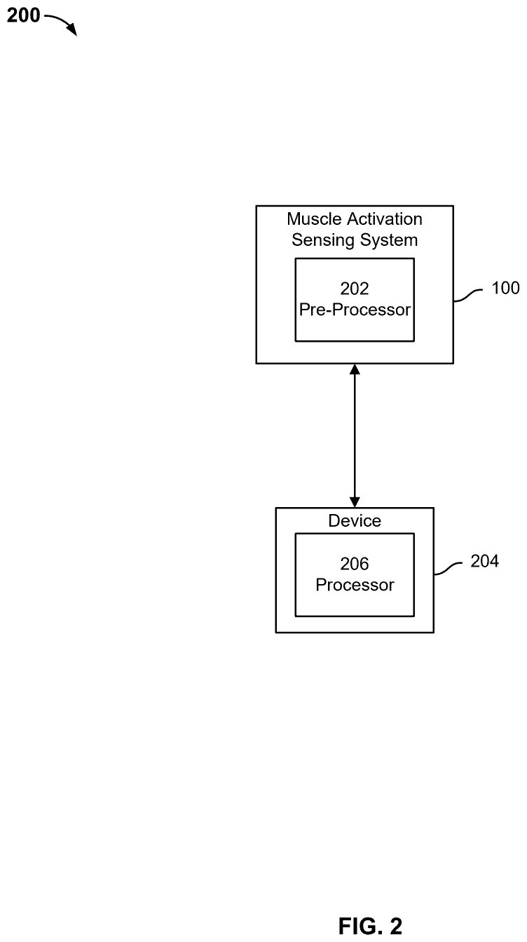

FIG. 2 is a block diagram of a control system in accordance with some embodiments. In the example shown, control system 200 includes a muscle activation sensing system 100 and a device 204 to be controlled. Device 204 may be a cell phone, a smart phone, a laptop, a desktop, a tablet, a smart watch, a smart television, a video game console, an electric vehicle, or any other electronic device capable of being controlled via an electronic signal. In some embodiments, the device 204 to be controlled is a device separate from processor 206.

Muscle activation sensing system 100 includes pre-processor 202. Pre-processor 202 is configured to convert the raw signals from a force sensor or a displacement sensor into specific actions. Pre-processor may provide one or more commands, instructions, or signals, to device 204 via a wired (e.g., tether) or wireless connection (e.g., Wi-Fi, Bluetooth, Bluetooth low energy (BLE), etc.). Pre-processor 202 may perform one or more pre-processing steps, such as filtering (e.g., low-pass filtering, high-pass filtering, band-pass filtering), offset correction, gain calibration/scaling, drift compensation, smoothing, windowing, etc.

Device 204 includes processor 206. In some embodiments, the device 204 to be controlled is a device separate from processor 206. In some embodiments, processor 206 is configured to perform post processing of the signals from muscle activation sensing system. For example, pre-processor 202 may perform signal cleanup and filtering, provide the cleaned up and filter signals to device 204. The processor 206 may interpret the received signals and control device 204 based on an interpretation of the received signals. In some embodiments, processor 206 utilizes artificial intelligence software to better interpret the muscle signals than pre-process 202. Interpreting the muscle signals is very processor intensive and may be better performed on device 204 than muscle activation sensing system 100 because device 204 has greater processing capabilities than muscle activation sensing system 100. In some embodiments, processor 206 performs pre-processing and post-processing on the sensor data.

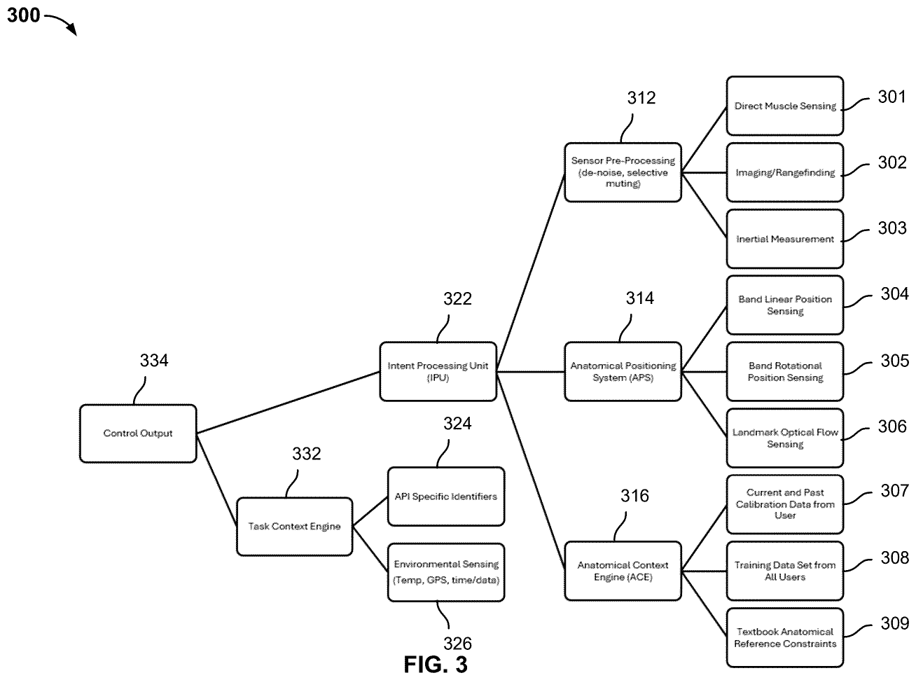

FIG. 3 is a block diagram of a muscle-based controller in accordance with some embodiments. In the example shown, muscle-based controller 300 may be included in a processor, such as processor 206.

Muscle-based controller 300 is configured to output a control output 334 to control or influence a device. Control output 334 is a signal or a command. Control output is based on an output of intent processing unit 322 and task context engine 332.

Intent processing unit 322 utilizes the sensor processed data 312, an output of anatomical positioning system 314, and an output of anatomical context engine 316 to determine an intent associated with a user's muscles.

Sensor Pre-Processing 312 is applied on raw sensor data to reduce the computational burden on higher-level algorithms. This may include noise reduction through filtering, as well as advanced techniques like selective muting—for example, temporarily disabling the force sensor when an accelerometer detects a brief period of rapid motion. In some embodiments, sensor pre-processing is performed by a muscle activation sensing system. In some embodiments, raw sensor data is outputted by a muscle activation sensing system and sensor pre-processing is performed by an external processor.

Sensor Pre-Processing 312 operates on data gathered from multiple sensing modalities to prepare it for higher-level processing. These include, but are not limited to: Direct Muscle Sensing 301, which captures signals directly from muscle activity; Imaging and Rangefinding 302, which uses optical or acoustic methods to detect the position and movement of the hand and arm; and Inertial Measurement 303, which relies on devices that measure acceleration and orientation. By combining and pre-processing these diverse data sources, the system reduces the computational load on downstream algorithms while preserving essential information for accurate control.

The Anatomical Positioning System (APS) 314 combines data from multiple sensors to estimate the position of the band on the arm. This includes Band Linear Position Sensing 304, which determines the band's position along the length of the arm, and Band Rotational Position Sensing 305, which measures the band's rotational orientation around the arm. Additionally, Landmark Optical Flow Sensing 306 uses optical or acoustic techniques to identify anatomical landmarks on or beneath the skin, providing further localization information to enhance positioning accuracy.

The Anatomical Context Engine (ACE) 316 integrates user-specific data and broader datasets with standardized anatomical models to generate maps of likely musculoskeletal relationships, each annotated with confidence levels. It draws on multiple sources, including current and historical calibration data from the user (current and past calibration data from user 307), aggregated training data from a wider user population (training data set from all users 308), and established anatomical reference models from textbooks and scientific literature (textbook anatomical reference constraints 309), to refine its estimations and improve accuracy.

The Task Context Engine (TCE) 332 adjusts system behavior based on what is being controlled and the surrounding environment, balancing signal reliability with the necessary control precision—for example, differentiating between the requirements of driving a wheelchair versus playing a video game. It leverages API-Specific Identifiers 324 to assess the current control target and determine which control elements should be treated as discrete (on/off) versus proportional (continuous). Additionally, it incorporates Environmental Sensing 326, which uses data from external sensors—whether on the band or in the environment—to interpret muscle activity in context, such as distinguishing between usage in a car versus on a basketball court.

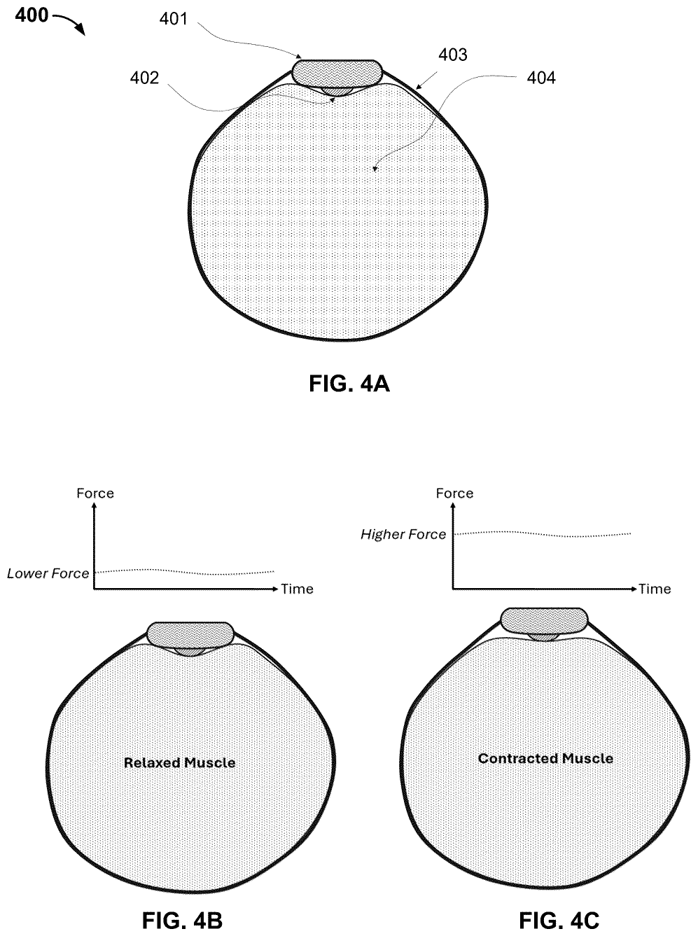

FIG. 4A is a block diagram illustrating an example of a muscle activation sensing system in accordance with some embodiments. In the example shown, muscle activation system 400 includes a housing 401 for a force or displacement sensor, a body part interface 402, a band 403 (rigid or flexible) for holding the sensor in contact with limb 404. Limb 404 may be an arm, leg, neck or any other body part.

A force sensor is mounted on the limb to detect changes in local muscle cross-sectional size and stiffness, which appear as variations in measured force. The sensor features a force or pressure-sensing element with a protruding geometry designed to improve sensitivity to muscle stiffness, size, and shape. It is positioned on the limb to provide a stable reaction force against the muscle, allowing it to capture meaningful mechanical changes as the muscle contracts or relaxes. Additionally, the sensor may be configured to measure either localized muscle displacement at the probe point or broader changes in overall limb shape and size. It can also be implemented as an array of multiple sensing elements, rather than a single sensor, to capture more detailed spatial information. Furthermore, the sensor may be designed as a multi-axis force sensor, capable of detecting both normal forces and shear forces for a more comprehensive understanding of muscle activity and tissue mechanics. This sensor leverages the natural signal processing capabilities of skeletal muscle tissue itself, providing superior performance compared to traditional EMG—offering higher signal-to-noise ratio and improved repeatability.

As shown in FIG. 4B, the force reading decreases when the muscle is relaxed. In contrast, as illustrated in FIG. 4C, muscle contraction causes the muscle to press against the force sensor, resulting in an increase in the force reading.

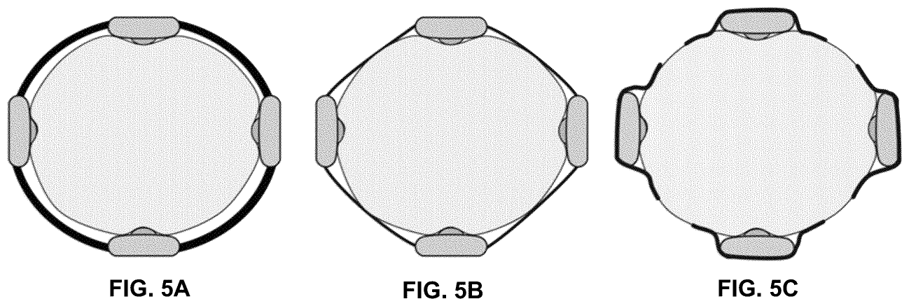

FIGS. 5A-5C are diagrams illustrating an arrangement and mounting of sensors in accordance with some embodiments.

Multiple sensors may be employed to expand the measurement coverage. These can be implemented as individual point sensors or integrated into a dense sensor array to achieve higher-fidelity data. While a single sensor is sufficient for basic operation, any number of sensors (e.g., 4, 8, 32, 100, or more) can be used depending on the application. Sensors may be positioned densely to provide redundant coverage over a target area or spaced strategically with non-uniform distribution to focus on specific areas of interest.

As shown in FIG. 5A, sensors can be mounted on a rigid or semi-rigid structure to maintain consistent spacing and orientation between sensors, as well as a repeatable distance and alignment relative to the limb. This fixed reference frame also simplifies the detection of limb size and shape changes by providing a stable baseline for comparison. Alternatively, as shown in FIG. 5B, sensors can be mounted on a flexible structure that conforms closely to the limb, offering a low-profile design. In this configuration, sensor readings reflect a combination of forces from both the limb (action force) and the structure (reaction force), effectively producing a weighted elastic average. Sensors may also be attached directly to the skin using adhesive or vacuum-based materials, which minimizes cross-talk by fully decoupling each sensor from the others.

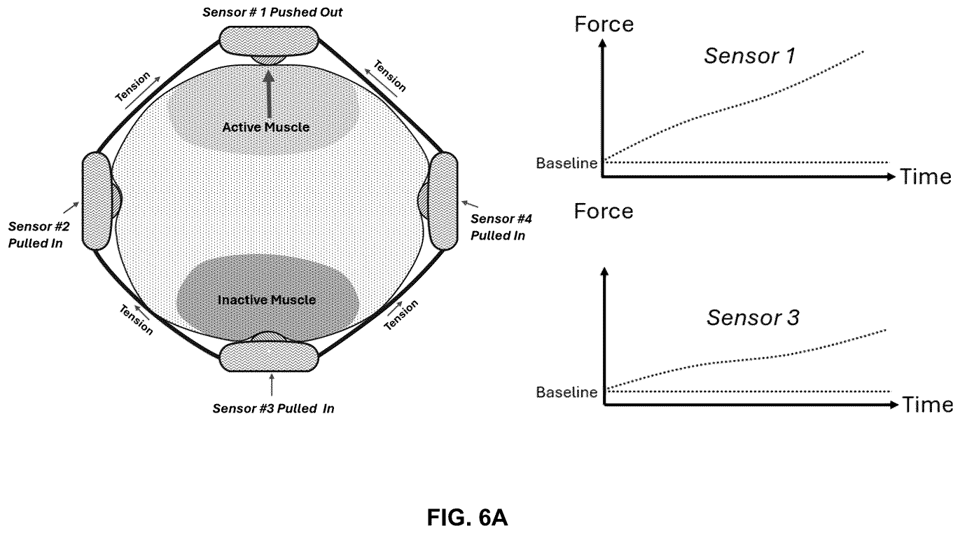

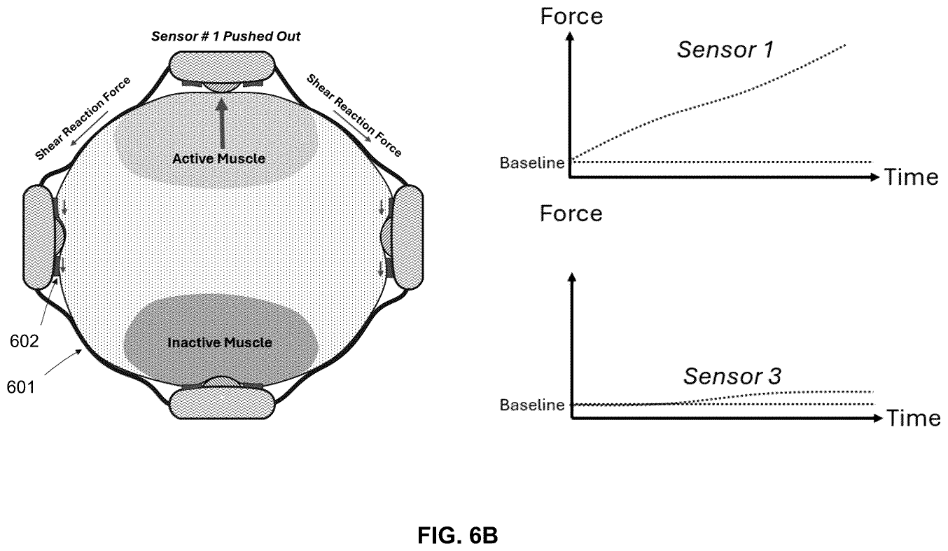

FIGS. 6A & 6B are diagrams illustrating crosstalk being reduced in the muscle activation sensing system in accordance with some embodiments. As illustrated in FIG. 6A, when a muscle on one side of the arm contracts and pushes outward, it increases tension in the strap around the arm. This tension can cause the sensor on the opposite side to be pulled inward, falsely indicating that the opposing muscle is active. In reality, this reading is the result of the strap transmitting force from the active muscle, not true activity from the opposing muscle. While shear (friction) forces between the strap and sensor help to limit the magnitude of this false signal, it still degrades measurement quality by introducing cross-talk between different muscle regions.

Two mechanical solutions can address this issue, both based on the principle of using friction to isolate each sensor from unwanted forces. The first is to enhance the capstan wrapping effect by designing the strap 601 with molded geometry that maintains greater contact with the skin, thereby improving shear force isolation for each sensor. The second is to increase the friction at the sensor itself by encapsulating or surrounding each sensor pad with a compliant, high-friction material 602 that provides sufficient surface engagement to prevent slippage without causing discomfort.

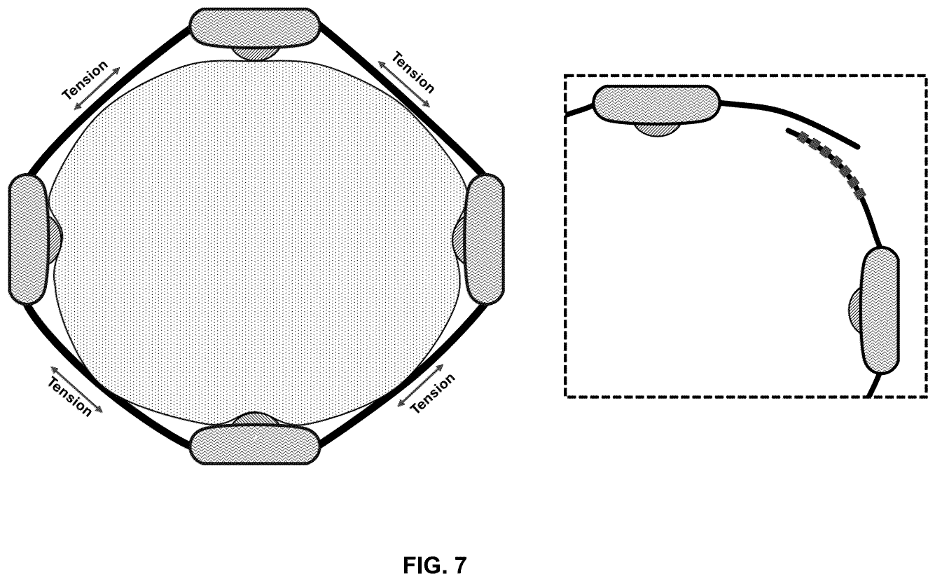

FIG. 7 is a diagram illustrating sensing limb size and shape in accordance with some embodiments. By measuring the tension or elongation of the band at one or more sensor locations, the total length or change in length of the band can be calculated. The band's initial, unloaded length can be determined by detecting the position of its length adjustment mechanism—either coarsely, using electrical sensing of discrete locking points such as holes or grooves, or continuously, using a magnetic, capacitive, or optical encoder. Since the initial length, the band's spring constant, and the measured force are all known, the current length of the band can be estimated at any time. This information can then be used to help determine the device's position on the arm.

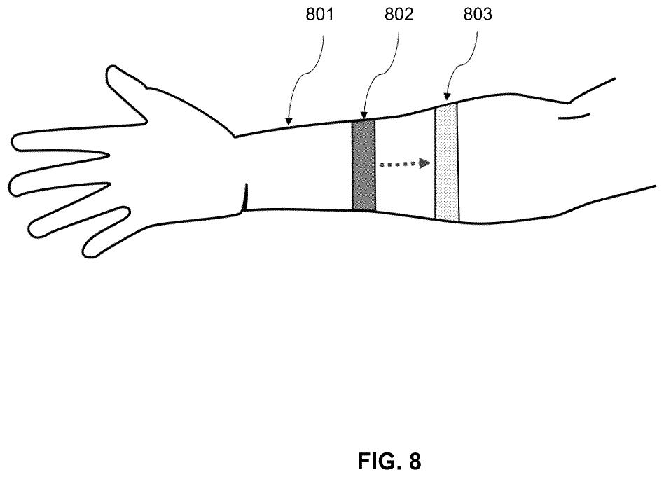

FIG. 8 is a diagram illustrating how the position of the Muscle Activation Sensing System on the arm can be determined in accordance with some embodiments. Knowing the length or tension in the band provides useful information for determining how the device is positioned on the arm. As illustrated in FIG. 8, the diagram shows the arm 801, the initial position 802 of the band on the arm, and the final position 803 after the band has shifted. When the band moves to a wider part of the arm 801, its circumference increases, and this change is detected electronically. Because the arm 801 naturally tapers, this measurement can be used to estimate the device's position along one axis of the arm 801.

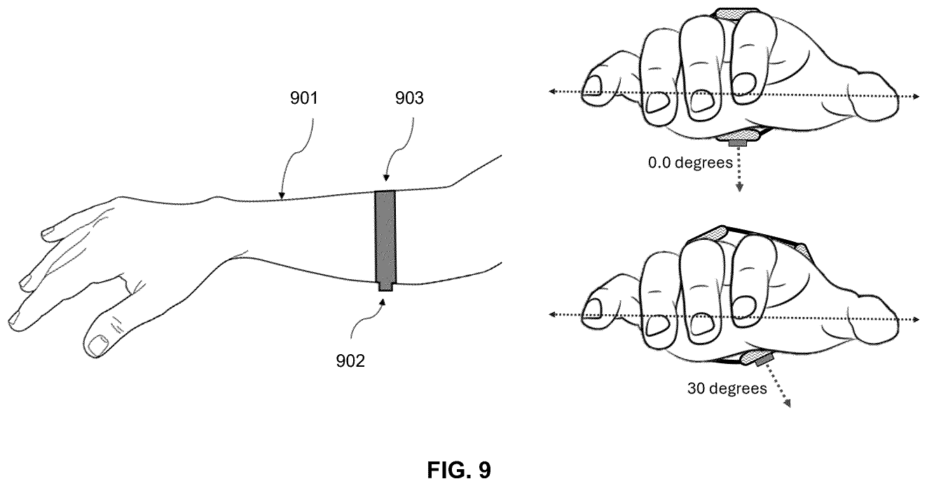

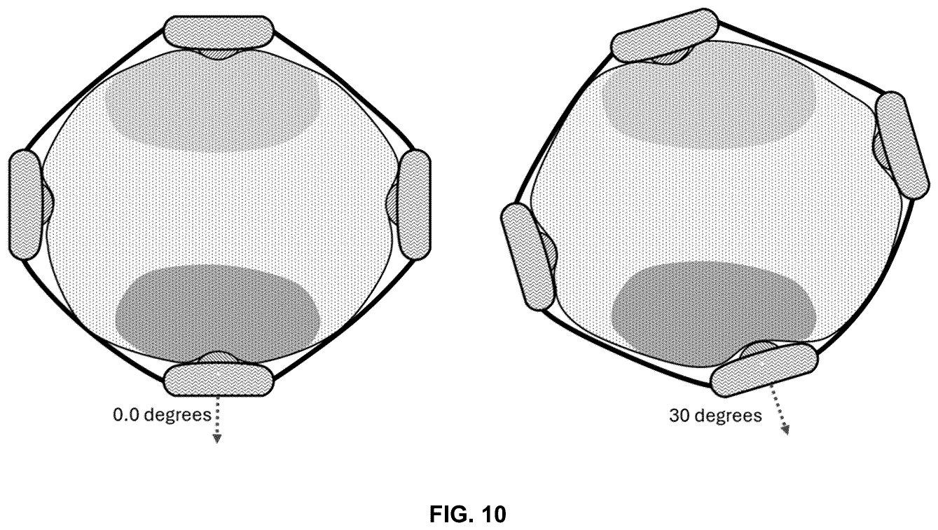

FIGS. 9 and 10 are diagrams illustrating how the band angle of the muscle activation sensing system can be determined in accordance with some embodiments.

By measuring the angle of the band (903) relative to Earth's gravitational acceleration using an accelerometer (902) or similar sensor, the rotational position of the band can be estimated. This is achieved by having the user move their arm 901 into a known reference posture—such as standing upright with the palm facing downward—while the device records the gravity vector. This allows the orientation of all sensors on the band to be mapped relative to the arm. The process involves four steps:
1. Place the band on the arm 901.
2. Position the arm 901 in a predefined posture, as guided by the device's software.
3. The accelerometer 902 measures the angle relative to gravity.
4. Using the known arm posture and the gravity measurement, the rotational position of the band 903 on the arm 901 is estimated.

Since the rotational position of the band 903 relative to the arm 901 has been established using the accelerometer 902, the orientation of each individual sensor in relation to specific areas of interest—such as targeted muscles—can be estimated. This positional context can then be used to improve the interpretation of muscle activity data. As illustrated in FIG. 10, gravity serves as a calibration reference, allowing the system to estimate the band's angle relative to the arm's anatomical structure.

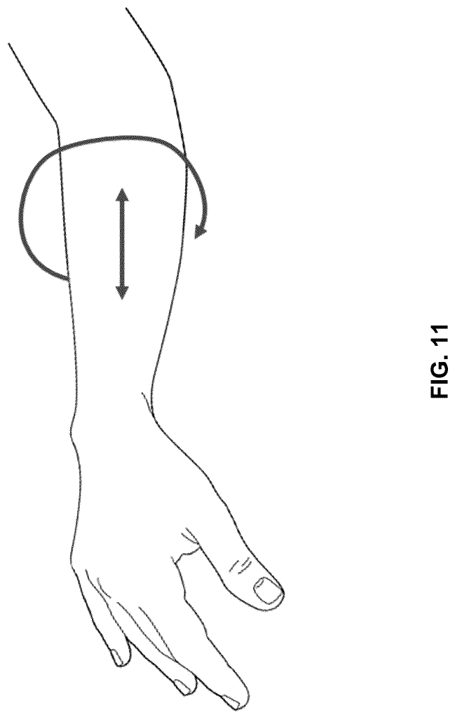

FIG. 11 is a diagram illustrating how a position of the muscle activation sensing system on the arm can be determined in accordance with some embodiments.

By measuring the band's length or tension, the system can estimate its position along the length of the arm. Additionally, by determining the band's angle relative to Earth's gravitational acceleration, its rotational orientation around the arm can be calculated. With both the linear and rotational positions known, the system can estimate the band's overall placement on the arm. This positional awareness enables dynamic reassociation of sensors to corresponding muscles, improving system performance while reducing the need for extensive training or calibration. Accurate sensor localization is a critical input to the Anatomical Context Engine (ACE), which leverages anatomical datasets to process sensor data more intelligently.

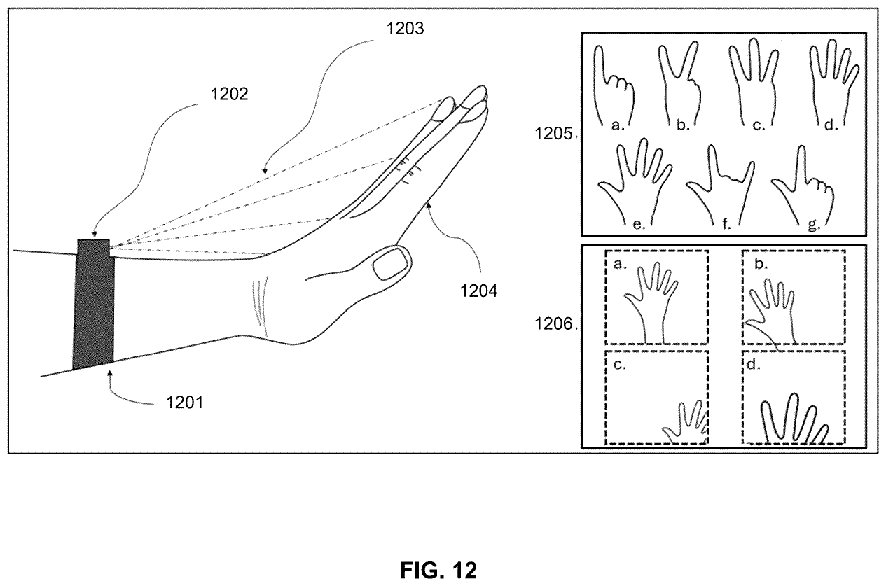

FIG. 12 is a diagram illustrating an imager in accordance with some embodiments. In the example shown, a camera or image sensor 1202 (e.g., visible, thermal, ToF, radar, LIDAR, etc.) is mounted on the band 1201 with its field of view 1203 covering the user's hand 1204 or arm. Illustration 1205 shows examples of hand gestures and orientations that the image sensor 1202 can detect. Illustration 1206 highlights various viewing perspectives captured by image sensor 102, which can be used to estimate the device's orientation and placement including pitch, roll, raw, and distance via size and blur.

This imaging capability serves two main purposes: first, to visually detect hand gestures and cross-reference them with other sensor data for improved accuracy, including identifying wrist poses such as flexion, extension, radial or ulnar deviation, and pronation or supination. Second, it helps determine the physical location of the device relative to the hand, supporting the Anatomical Positioning System (APS) and enhancing the contextual awareness and performance of other sensors.

This imaging setup not only provides additional gesture context to support software algorithms but also enables real-time estimation of the device's position. This positional awareness allows the system to dynamically adjust how it interprets data from other sensors, reducing the need for repeated calibration and improving overall performance.

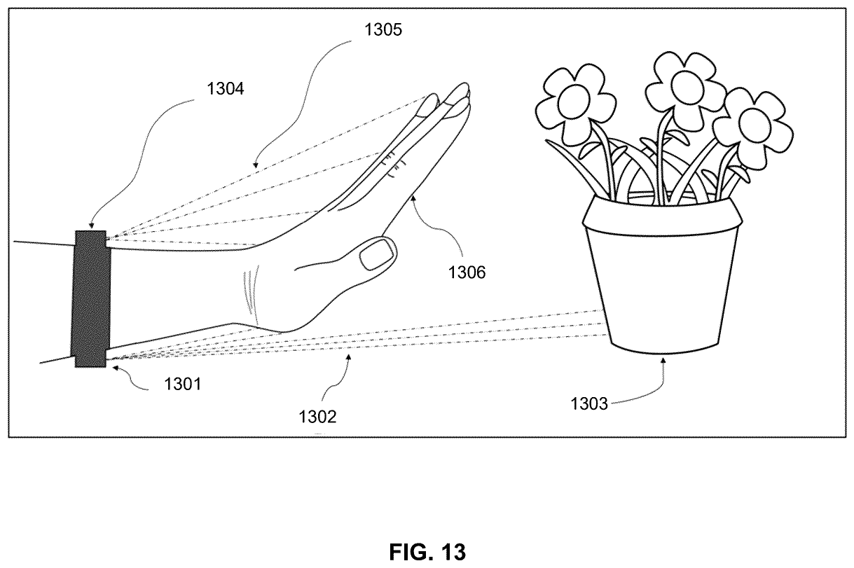

FIG. 13 is a diagram illustrating an imager and distance sensor in accordance with some embodiments. In the example shown, the band includes a distance sensor 1301 having a field of view 1302. The distance sensor 1301 may output a value indicating a distance between the distance sensor and object 1303. The band includes a camera or image sensor 1304 (e.g., visible, thermal, ToF, radar, LIDAR, etc.) with its field of view 1305 covering the user's hand 1204 or arm.

One or more distance sensors 1301, such as Time-of-Flight (ToF) sensors, can be mounted on the band with the hand 1306 or arm positioned within their field of view. These sensors serve three primary functions. First, they enable the detection of hand gestures and wrist poses—such as flexion, extension, radial or ulnar deviation, and pronation or supination—providing additional context that can be cross-referenced with other sensor data for improved accuracy. Second, they help determine the device's physical position relative to the hand, supporting the Anatomical Positioning System (APS) to enhance the interpretation and performance of other sensors. Third, they allow the system to detect additional objects in the field of view and provide distance information that helps the image processing algorithms isolate the correct anatomical features—such as the hand or arm—from background noise. Whether implemented as a multi-zone array (e.g., an 8×8 imager) or as a set of single-point measurements, these distance sensors improve the system's ability to filter out irrelevant visual or geometric noise, resulting in more accurate and efficient data processing.

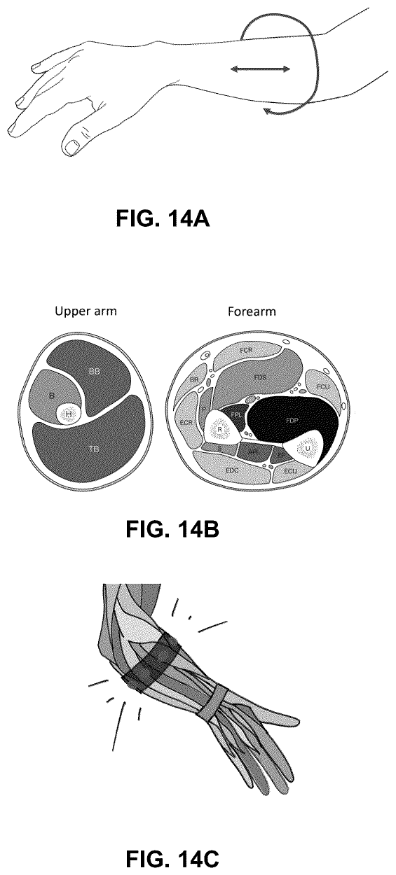

FIGS. 14A-C are diagrams illustrating how using anatomical information can be used to aid in algorithm interpretation of user intent in accordance with some embodiments.

As shown in FIG. 14A, by knowing the band's location or zone on the arm, the system can estimate its overall position relative to specific muscles. This positional awareness allows the device to dynamically reassociate sensors with corresponding muscles, improving performance while reducing the need for repeated training or calibration. Additionally, as illustrated in FIG. 14B, anatomical datasets can be used to enhance sensor data interpretation. For example, the system can reference known muscle sizes to distinguish whether a large signal comes from a small muscle moving significantly or from a larger muscle moving slightly. Similarly, detecting movement in a primary muscle may indicate a larger overall motion. If the system can also identify the current posture of the hand, it can predict which muscles are likely to be active based on established anatomical relationships. Finally, as shown in FIG. 14C, this anatomical context enables the system to interpret sensor data more intelligently, based on the expected functional behavior of the muscles.

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. A system, comprising:
a muscle activation sensing system that includes a mechanical sensor mounted on a body part and configured to detect a change in a muscle associated with the body part;
a processor configured to:
determine a location of the muscle activation sensing system on the body part;
derive a control signal based on the detected change in the muscle associated with the body part and the determined location; and
output the control signal to a device.

2. The system of claim 1, wherein the mechanical sensor is a force sensor.

3. The system of claim 1, wherein the mechanical sensor is a displacement sensor.

4. The system of claim 1, wherein the muscle activation sensing system includes a housing, wherein the housing includes a body part interface to contact a skin of the body part.

5. The system of claim 4, wherein the body part interface is encapsulated or surrounded by a high-friction material.

6. The system of claim 1, wherein the muscle activation sensing system includes a band.

7. The system of claim 6, wherein the band is rigid or flexible.

8. The system of claim 6, wherein the band has a molded geometry.

9. The system of claim 1, wherein an output of the mechanical sensor is pre-processed including by filtering, offset correction, gain calibration or scaling, drift compensation, smoothing, or windowing.

10. The system of claim 1, wherein the control signal is derived based on outputs of an intent processing unit and a task context engine.

11. The system of claim 10, wherein an output of the intent processing unit is based on corresponding outputs associated with pre-processed sensor data, corresponding output associated with an anatomical positioning system, and a corresponding output associated with an anatomical context engine.

12. The system of claim 10, wherein an output of the task context engine is based on API specific identifiers and environmental sensing.

13. The system of claim 1, wherein the muscle activation sensing system includes the mechanical sensor and one or more other mechanical sensors.

14. The system of claim 13, wherein a tension between the mechanical sensor and one of the one or more other mechanical sensors is used to determine the location of the muscle activation sensing system.

15. The system of claim 1, wherein the processor is configured to determine a rotational position of the mechanical sensor by the body part moving into a known reference posture and recording a gravity vector while the body part is moved into the known reference posture.

16. The system of claim 1, wherein the muscle activation sensing system includes an image sensor configured to detect gestures associated with a hand.

17. The system of claim 16, wherein the detected gestures associated with the hand are utilized in part to derive the control signal.

18. The system of claim 1, wherein the muscle activation sensing system includes a distance sensor.

19. The system of claim 18, wherein an output of the distance sensor is utilized in part to derive the control signal.

20. A method, comprising:
detecting, by a muscle activation sensing system that includes a mechanical sensor mounted on a body part, a change in a muscle associated with the body part;
determining a location of the muscle activation sensing system on the body part;
deriving a control signal based on the detected change in the muscle associated with the body part and the determined location; and
outputting the control signal to a device.

21. The method of claim 20, wherein the mechanical sensor is a force sensor.

22. The method of claim 20, wherein the mechanical sensor is a displacement sensor.

23. A computer program product embodied in a non-transitory computer readable medium and comprising computer instructions for:
detecting, by a muscle activation sensing system that includes a mechanical sensor mounted on a body part, a change in a muscle associated with the body part;
determining a location of the muscle activation sensing system on the body part;
deriving a control signal based on the detected change in the muscle associated with the body part and the determined location; and
outputting the control signal to a device.
